(19) United States
(12) Patent Application Publication
(10) Pub. No.: US 2010/0036482 A1
(43) Pub. Date: Feb. 11, 2010

(54) DRUG DELIVERY SYSTEM AND METHOD OF MANUFACTURING THEREOF

(75) Inventors: Richard C. Svrluga, Newton, MA (US); Sean R. Kirkpatrick, Littleton, MA (US)

Correspondence Address: BURNS & LEVINSON, LLP, 125 SUMMER STREET, BOSTON, MA, 02110, US

(73) Assignee: Exogenesis Corporation, Billerica, MA (US)

(21) Appl. No.: 12/537,388

(22) Filed: Aug. 7, 2009

Related U.S. Application Data
(60) Provisional application No. 61/086,981, filed on Aug. 7, 2008.

(51) Int. Cl. A61F 2/82; A61F 2/30; A61N 1/36; B05D 3/06; B05D 7/00

(52) U.S. Cl. 623/1.42; 623/18.11; 607/9; 427/2.25; 427/2.26

(57) ABSTRACT

A medical device for surgical implantation adapted to serve as a drug delivery system has one or more drug loaded holes with barrier layers to control release or elution of the drug from the holes or to control inward diffusion of fluids into the holes. The barrier layers are non-polymers and are formed from the drug material itself by ion beam processing. The holes may be in patterns to spatially control drug delivery. Flexible options permit combinations of drugs, variable drug dose per hole, multiple drugs per hole, temporal control of drug release sequence and profile. Methods for forming such a drug delivery system are also disclosed.

### CROSS REFERENCE TO RELATED APPLICATIONS

This application claims priority from U.S. Provisional Patent Application Ser. No. 61/086,981, filed Aug. 7, 2008 and incorporated herein by reference in its entirety.

### FIELD OF THE INVENTION

This invention relates generally to drug delivery systems such as, for example, medical devices implantable in a mammal (e.g., coronary and/or vascular stents, implantable prostheses, etc.), and more specifically to a method and system for applying drugs to the surface of medical devices and for controlling the surface characteristics of such drug delivery systems such as, for example, the drug release rate and bio-reactivity, using ion beam technology, preferably gas cluster ion beam (GCIB) technology in a manner that promotes efficacious release of the drugs from the surface over time.

### BACKGROUND OF THE INVENTION

Medical devices intended for implant into or for direct contact with the body or bodily tissues of a mammal (including a human), as for example medical prostheses or surgical implants, may be fabricated from a variety of materials including various metals, metal alloys, plastic, polymer, or co-polymer materials, solid resin materials, glassy materials and other materials as may be suitable for the application and appropriately biocompatible. As examples, certain stainless steel alloys, cobalt-chrome alloys, titanium and titanium alloys, biodegradable metals like iron and magnesium, polyethylene and other inert plastics have been used. Such devices include for example, without limitation, vascular stents, artificial joint prostheses (and components thereof), coronary pacemakers, etc. Implantable medical devices are frequently employed to deliver a drug or other biologically active beneficial agent to the tissue or organ in which it is implanted.

A coronary or vascular stent is just one example of an implantable medical device that has been used for localized delivery of a drug or other beneficial agent. Stents may be inserted into a blood vessel, positioned at a desired location and expanded by a balloon or other mechanical expansion device. Unfortunately, the body's response to this procedure often includes thrombosis or blood clotting and the formation of scar tissue or other trauma-induced tissue reactions at the treatment site. Statistics show that restenosis or re-narrowing of the artery by scar tissue after stent implantation occurs in a substantial percent of the treated patients within only six months after these procedures, leading to severe complications in many patients.

Coronary restenotic complications associated with stents are believed to be caused by many factors acting alone or in combination. These complications can be reduced by several types of drugs introduced locally at the site of stent implantation. Because of the substantial financial costs associated with treating the complications of restenosis, such as catheterization, re-stenting, intensive care, etc., a reduction in restenosis rates would save money and reduce patient suffering.

There are many current popular designs of coronary and vascular stents. Although the use of coronary stents is growing, the benefits of their use remain controversial in certain clinical situations or indications due to their potential complications. It is widely held that during the process of expanding the stent, damage occurs to the endothelial lining of the blood vessel triggering a healing response that re-occludes the artery. To help combat that phenomenon, drug-bearing stents have been introduced to the market to reduce the incidence of restenosis or re-occluding of the blood vessel. These drugs are typically applied to the stent surface or mixed with a liquid polymer or co-polymer that is applied to the stent surface and subsequently hardens. When implanted, the drug elutes out of the polymeric mixture in time, releasing the medicine into the surrounding tissue. There remain a number of problems associated with this technology. Because the stent is expanded at the diseased site, the polymeric material has a tendency to crack and sometimes delaminate from the stent surface. These polymeric flakes can travel throughout the cardio-vascular system and cause significant damage. There is evidence to suggest that the polymers themselves cause a toxic reaction in the body. Additionally, because of the thickness of the coating necessary to carry the required amount of medicine, the stents can become somewhat rigid making expansion difficult. Also, because of the volume of polymer required to adequately contain the medicine, the total amount of medicine that can be loaded may be undesirably reduced.

In other prior art stents, the bare wire or metal mesh of the stent itself is coated with one or more drugs through processes such as high pressure loading, spraying, and dipping. However, loading, spraying and dipping do not always yield the optimal, time-release dosage of the drugs delivered to the surrounding tissue. The drug or drug/polymer coating can include several layers such as the above drug-containing layer as well as a drug-free encapsulating layer, which can help to reduce the initial drug release amount caused by initial exposure to liquids when the device is first implanted.

A variety of methods have been employed to attach drugs or other therapeutic agents to an implantable medical device and to control the release rate of the drug/agent after surgical implantation. An example includes providing holes in the surface of the implantable medical device. These holes are filled with the desired drug or agent or combinations thereof. U.S. Pat. No. 7,208,011 issued to Shanley et al. discloses the use of drug-filled holes in a coronary stent. Barrier layers of polymers or co-polymers are formed at the bottoms and/or tops of the holes to control the release rates of the attached drugs/agents and/or to control the rate of diffusion of external fluids (such as water or biological fluids) into the attached drugs. Drug/polymer mixtures are also employed in filling the holes. The use of holes to contain the drug increases the amount of drug that can be retained on the stent and also reduces the amount of undesirable polymer or co-polymer that is required. However, as previously explained, these polymers or co-polymers, while contributing to the control of the drug release rate, can have undesirable characteristics that reduce the over medical success of the drug loaded implantable device and it is desirable that they could be completely eliminated.

Gas cluster ion beams have been employed to smooth or otherwise modify the surfaces of implantable medical devices such as stents and other implantable medical devices. For example, U.S. Pat. No. 6,676,989C1 issued to Kirkpatrick et al. teaches a GCIB processing system having a holder and manipulator suited for processing tubular or cylindrical workpieces such as vascular stents. In another example, U.S. Pat. No. 6,491,800B2 issued to Kirkpatrick et al. teaches a GCIB processing system having workpiece holders and manipulators for processing other types of non-planar medical devices, including for example, hip joint prostheses. In still another example, U.S. Pat. No. 7,105,199B2 issued to Blinn et al. teaches the use of GCIB processing to improve the adhesion of drug coatings on stents and to modify the elution or release rate of the drug from the coatings.

In view of this new approach to in situ drug delivery, it is desirable to have the greater drug loading capacity provided by the use of holes, while reducing or eliminating the necessity of employing a polymer material to bind the drug and/or control its release or elution rate from the implantable device as well as control over other surface characteristics of the drug delivery medium.

It is therefore an object of this invention to provide a means of applying substantial quantities of drugs to medical devices and controlling the elution or release rate without requiring the incorporation of polymers by using ion beam technology, preferably gas cluster ion beam technology.

It is a further object of this invention to transform the surfaces of medical devices into drug delivery systems by providing holes for drug retention and treating the surfaces of the drugs with an ion beam, preferably a gas cluster ion beam so as to facilitate a timed release of the drug(s) from the surfaces.

Yet another object of this invention to transform the surfaces of medical devices into drug delivery systems by providing holes for drug retention and treating the surfaces of the drugs with an ion beam, preferably a gas cluster ion beam so as to retard the diffusion of an external (water or biological) fluid into the retained drug.

Still another object of this invention is to provide a medical device that is a drug delivery system for delivering a substantial quantity of a drug with spatial and temporal control of the drug delivery.

### SUMMARY OF THE INVENTION

The objects set forth above as well as further and other objects and advantages of the present invention are achieved by the invention described herein below.

The present invention is directed to the use of holes in a medical device for containing a drug, the introduction of drugs into the holes for containment therein, and the use of ion beam processing, preferably GCIB processing, to modify the surface of the contained drug to modify a surface layer of the contained drug so as to control the rate at which the drug or agent is released or eluted and/or to control the rate at which external fluids penetrate through the surface layer to the underlying drug, thereby eliminating the need for a polymer, co-polymer or any other binding agent and transforming the medical device surface into a drug delivery system. This will prevent the problem of toxicity and the damage caused by transportation of delaminated polymeric material throughout the body. Unlike the above-described prior art stents that contain drug-filled holes and utilize a separately applied polymer barrier layer material or a drug-polymer (or co-polymer) mixture to control drug release or elution rate, the present invention provides the ability to completely avoid the use of a polymer or co-polymer binder or barrier layer in the preparation of a drug-releasing implantable medical device.

Beams of energetic conventional ions, electrically charged atoms or molecules accelerated through high voltage fields, are widely utilized to form semiconductor device junctions, to smooth surfaces by sputtering, and to enhance the properties of thin films. Unlike conventional ions, gas cluster ions are formed from clusters of large numbers (having a typical distribution of several hundreds to several thousands with a mean value of a few thousand) of weakly bound atoms or molecules of materials (that are gaseous under conditions of standard temperature and pressure—commonly inert gas such as argon, for example) sharing common electrical charges and which are accelerated together through high voltages (on the order of from about 3 to 70 kY or more) to have high total energies. Being loosely bound, gas cluster ions disintegrate upon impact with a surface and the total energy of the cluster is shared among the constituent atoms. Because of this energy sharing, the atoms are individually much less energetic than the case of conventional ions or ions not clustered together and, as a result, the atoms penetrate to much shorter depths.

Because the energies of individual atoms within an energetic gas cluster ion are very small, typically a few eV to some tens of eV, the atoms penetrate through only a few atomic layers, at most, of a target surface during impact. This shallow penetration (typically a few nanometers to about ten nanometers, depending on the beam acceleration) of the impacting atoms means all of the energy carried by the entire cluster ion is consequently dissipated in an extremely small volume in the top surface layer during a time period less than a microsecond. This is different from using conventional ion beams where the penetration into the material is sometimes several hundred nanometers, producing changes deep below the surface of the material. Because of the high total energy of the gas cluster ion and extremely small interaction volume, the deposited energy density at the impact site is far greater than in the case of bombardment by conventional ions. For this reason, the GCIB is capable of interacting with the surface of an organic material like a drug to produce profound changes in a very shallow surface layer of about 10 nanometers of less. Such changes may include cross linking of molecules, densification of the surface layer, carbonization of organic materials in the surface layer, polymerization, and other forms of denaturization.

GCIBs are generated and transported for purposes of irradiating a workpiece according to known techniques as taught for example in the published U.S. Patent Application 2009/0074834A1 by Kirkpatrick et al., the entire contents of which are incorporated herein by reference.

As used herein, the term “drug” is intended to mean a therapeutic agent or a material that is active in a generally beneficial way, which can be released or eluted locally in the vicinity of an implantable medical device to facilitate implanting (for example, without limitation, by providing lubrication) the device, or to facilitate (for example, without limitation, through biological or biochemical activity) a favorable medical or physiological outcome of the implantation of the device. “Drug” is not intended to mean a mixture of a drug with a polymer that is employed for the purpose of binding or providing coherence to the drug, attaching the drug to the medical device, or for forming a barrier layer to control release or elution of the drug. A drug that has been modified by ion beam irradiation to densify, carbonize or partially carbonize, partially denature, cross-link or partially cross-link, or to at least partially polymerize molecules of the drug is intended to be included in the “drug” definition.

As used herein, the term “polymer” is intended to include co-polymers and to mean a material that is significantly polymerized and which is not biologically active in a generally beneficial way in either its monomer or polymer form. Typical polymers may include, without limitation, polylactic acid, polyglycolic acid, polylactic-co-glycolic acid, polylactic acid-co-caprolactone, polyethylene glycol, polyethylene oxide, polyvinyl pyrrolidone, polyorthoesters, polysaccharides, polysaccharide derivatives, polyhyaluronic acid, polyalginic acid, chitin, chitosan, various celluloses, polypeptides, polylysine, polyglutamic acid, polyanhydrides, polyhydroxy alkonoates, polyhydroxy valerate, polyhydroxy butyrate, and polyphosphate esters. The term “polymer” is not intended to include a drug that has been modified by ion beam irradiation to densify, carbonize or partially carbonize, partially denature, cross-link or partially cross-link, or to at least partially polymerize molecules of the drug.

As used herein, the term “hole” is intended to mean any hole, cavity, crater, trough, trench, or depression penetrating a surface of an implantable medical device and may extend through a portion of the device (through-hole), or only part way through the device (blind-hole, or cavity) and may be substantially cylindrical, rectangular, or of any other shape.

The application of the drug(s) to the medical device may be accomplished by several methods. The surface of the medical device, which may be composed, for example, of a metal, metal alloy, ceramic, or any other non-polymer material, is first processed to form one or more holes in the surface thereof. The desired drug(s) is then deposited into the holes. The drug deposition (hole loading) may be by any of numerous methods, including spraying, dipping, electrostatic deposition, ultrasonic spraying, vapor deposition, or by discrete droplet-on-demand fluid jetting technology. When spraying, dipping, electrostatic deposition, ultrasonic spraying, vapor deposition, or similar techniques are employed, a conventional masking scheme may be employed to limit deposition to selected locations. Discrete droplet-on-demand fluid-jetting is a preferred method because it provides the ability to introduce precise volumes of liquid drugs or drugs-in-solution into precisely programmable locations. Discrete droplet-on-demand fluid jetting may be accomplished using commercially available fluid-jet print head jetting devices as are available (for example, not limitation) from MicroFab Technologies, Inc., of Plano, Tex.

After the holes have been drug-loaded, the present invention uses ion beam irradiation, preferably GCIB irradiation, to modify a very shallow surface layer of the retained drug to alter the drug in that layer in a way that modifies its properties in a way that forms a thin surface film with barrier properties that limit diffusion across the surface film. This results in the ability to control the rate of diffusion of water or other biological fluids into the drug retained in the hole, and to control the rate of elution of the drug out from the hole. The modification of the surface portion of the drug that becomes the surface film having barrier properties may consist of any of several modification outcomes depending on the nature of the drug, and the nature of the ion beam (preferably GCIB) processing. Possible outcomes include cross-linking or polymerizing of the drug molecules, carbonization of the drug material by driving out more volatile atoms from the molecules, densification of the drug, and other forms of denaturization that result in reduced solubility, erodibility, and/or in reduced porosity or diffusion rates.

The application of drugs via GCIB surface modification such as described above will reduce complications, lead to genuine cost savings and an improvement in patient quality of life, and overcome prior problems of thrombosis and restenosis, Preferred therapeutic agents for delivery in the drug delivery systems of the present invention include anti-coagulants, antibiotics, immunosuppressant agents, vasodilators, anti-prolifics, anti-thrombotic substances, anti-platelet substances, cholesterol reducing agents, anti-tumor medications and combinations thereof.

### BRIEF DESCRIPTION OF THE DRAWINGS

For a better understanding of the present invention, together with other and further objects thereof, reference is made to the accompanying drawings, wherein:

FIG. 1A is a coronary stent with through-holes as may be employed in embodiments of the invention. FIG. 1B is a second view of the coronary stent simplified for clarity by removal of detail beyond the nearest surface;

FIG. 2 is a view of coronary stent with blind-holes as may be employed in embodiments of the invention;

FIGS. 3A, 3B, and 3C are views of prior art holes in prior art stents, illustrating various prior art loading of holes by employing polymers;

FIGS. 4A, 4B, 4C, and 4D show steps in the formation of a drug loaded through-hole in a stent according to an embodiment of the invention;

FIGS. 5A, 5B, and 5C show steps in the formation of a drug loaded blind-hole in a stent according to an embodiment of the invention;

FIGS. 6A and 6B show optional steps for GCIB processing of a hole edge according to an embodiment of the invention; and

FIG. 7 shows a cross section view of a portion of a surface of an implantable medical device, illustrating the variety of methods that can be employed within the present invention to control drug administration.

### DETAILED DESCRIPTION OF THE DRAWINGS

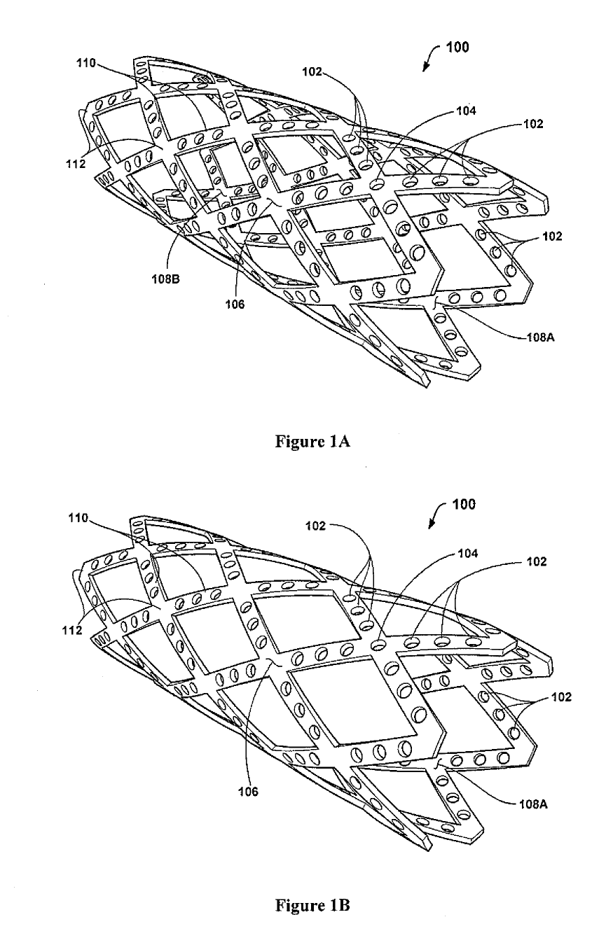

Reference is now made to FIG. 1A is a perspective view of an expandable coronary stent 100 with through-holes as may be employed in embodiments of the invention. It is understood by the inventors that the present invention is applicable to a wide variety of implantable medical devices, but for explanatory purposes, the stent 100 is shown as an example. Stent 100 is an expandable metal coronary stent shown in an expanded, or partially expanded state. Expandable stents are manufactured in many configurations each having various advantages and disadvantages. The configuration shown in FIG. 1A is a simple diamond-shaped mesh shown not for limitation but to simplify explanation of the present invention. The stent 100 has struts (110 for examples) and intersections (112 for examples) that join the struts 110. The stent 100 has an inner surface (indicated as 108A and 108B, for example) forming the lumen of the stent and an outer surface (indicated as 106) forming the vascular scaffold. Holes (102 for examples) may be located in the struts. Other holes (104 for example) may be located in the intersections. The holes 102 and 104 are through-holes, penetrating from the outer surface 106 to the inner surface 108A and 108B. The struts 110 and intersections 112 are pointed out to illustrate the common fact that stents of diverse configurations may have differing regions that may be differently affected when the stent is expanded. For example, in the stent 100, as illustrated here, certain holes 102 located near the intersections 112 may experience more strain during expansion than will holes 104 in the intersections and other holes 102 located further from the intersections 112. It will be apparent to those skilled in the art that stents of other configurations may have locations where holes will experience greater or lesser degrees of strain during expansion. In FIG. 1A, the holes 102, 104 are shown as having a relatively large diameter in comparison to the dimensions of the struts 110 and intersections 112. These relative sizes are chosen for clarity of illustration of the concept and are not intended to be limiting of the invention. It will be appreciated by those skilled in the art that holes of smaller relative diameters than those illustrated may experience smaller degrees of strain during expansion of the stent than that experienced by the larger holes as illustrated. It will be appreciated by those skilled in the arts that the holes could be any of a variety of sizes and patterns and in differing locations relative to features of the stent and still be within the spirit of the invention. FIG. 1A represents a stent that is similar to prior art stents and that is also suitable for illustrating the present invention.

FIG. 1B is a second view of the expandable coronary stent 100. It is the identical stent, but the drawing is simplified for clarity by removal of detail beyond the nearest surface. That is to say, the portion 108B of the inner surface, which is behind the nearer portions of the stent 100, has been removed from the drawing to simplify and clarify it, while the portion of the inner surface 108A remains in the drawing. FIG. 1B represents a stent that is similar to prior art stents and that is also suitable for illustrating the present invention. According to the present invention, the holes 102, 104 may be formed by any practical method including laser machining or by focused ion beam machining.

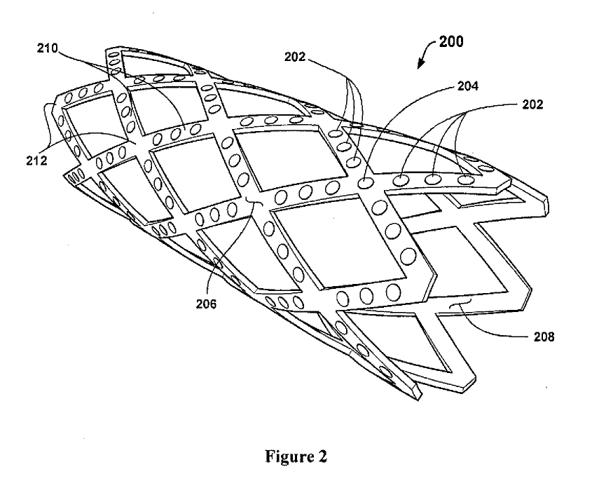

FIG. 2 is a perspective view of an expandable coronary stent 200 with blind-holes as may be employed in embodiments of the invention. The drawing is simplified for clarity by removal of detail beyond the nearest surface. Stent 200 is an expandable metal coronary stent shown in an expanded, or partially expanded state. The stent 200 has struts (210 for examples) and intersections (212 for examples) that join the struts 210. The stent 200 has an inner surface 208 forming the lumen of the stent and an outer surface 206 forming the vascular scaffold. Holes (202 for examples) may be located in the struts. Other holes (204 for example) may be located in the intersections. The holes 202 and 204 are blind-holes, not penetrating from the outer surface 206 to the inner surface 208, but rather penetrating only part of the way through the thickness of the stent wall. The holes 202, 204 are shown as having a relatively large diameter in comparison to the dimensions of the struts 210 and intersections 212. These relative sizes are chosen for clarity of illustration of the concept and are not intended to be limiting of the invention. It will be appreciated by those skilled in the art that holes of smaller relative diameters than those illustrated may experience smaller degrees of strain during expansion of the stent than that experienced by the larger holes as illustrated. It will be appreciated by those skilled in the arts that the holes could be any of a variety of sizes, patterns, depths, and in differing locations relative to features of the stent and still be within the spirit of the invention.

FIG. 2 represents a stent that is similar to prior art stents and that is also suitable for illustrating the present invention. According to the present invention, the holes 202, 204 may be formed by any practical method including laser machining or by focused ion beam machining.

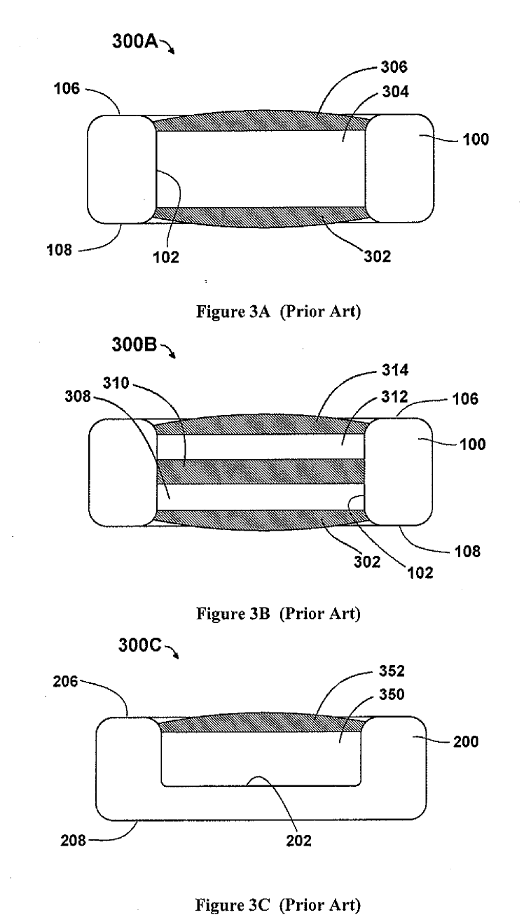

FIG. 3A shows a sectional view 300A of a prior art hole 102 in prior art stent 100, illustrating a prior art method of loading a hole with a drug by employing polymers. A therapeutic layer 304 consists of a drug or a drug-polymer mixture. A barrier layer 302 on the inner surface 108 of the stent 100 comprises a polymer and prevents elution or controls the elution rate of the therapeutic layer 304 to the inner portion (lumen) of the stent. A second barrier layer 306 on the outer surface 106 of the stent 100 comprises a polymer and controls the elution rate of the therapeutic layer 304 to the outer portion (vascular scaffold) of the stent. The barrier layers 302 and 306 may also control or prevent the diffusion of water or other biological fluids from outside of the stent into the therapeutic layer 304 retained by the hole in the stent. The barrier layers 302 and 306 may be biodegradable or erodible materials comprising polymer to provide a delayed release of the enclosed therapeutic layer 304. The therapeutic layer 304 may be a drug or alternatively may be a mixture of drug and polymer to further delay or control the elution or release rate of the therapeutic layer 304.

FIG. 3B shows a sectional view 300B of a prior art hole 102 in prior art stent 100, illustrating a prior art method of loading a hole with multiple layers of a drug by employing polymers. Therapeutic layers 308, 312 consist respectively a drug or a drug-polymer mixture and may comprise similar or dissimilar drugs. Barrier layer 302 on the inner surface 108 of the stent 100 comprises a polymer and prevents elution or controls the elution rate of the therapeutic layer 308 to the inner portion (lumen) of the stent. A second barrier layer 314 on the outer surface 106 of the stent 100 comprises a polymer and controls the elution rate of the therapeutic layer 312 to the outer portion (vascular scaffold) of the stent. A third barrier layer 310 may comprise polymer and separates the therapeutic layers 308 and 312 and may also prevent the elution or control the elution rate of the therapeutic layers 308 and 310. The barrier layers 302, 310 and 314 may also control or prevent the diffusion of water or other biological fluids from outside of the stent into the therapeutic layers 308 and 312 retained by the hole in the stent. The barrier layers 302, 310, and 314 may be biodegradable or erodible materials comprising polymer to provide a delayed release of the enclosed therapeutic layers 308 and 312. The therapeutic layers 308 and 312 may be each be either a drug or alternatively may be a mixture of drug and polymer to further delay or control the elution or release rate of the therapeutic layers 308 and 312.

FIG. 3C shows a sectional view 300C of a prior art blind-hole 202 in a prior art stent 200, illustrating a prior art method of loading a hole with a drug by employing polymers. A therapeutic layer 350 consists of a drug or a drug-polymer mixture. A barrier layer 352 on the outer surface 206 of the stent 200 comprises a polymer and controls the elution rate of the therapeutic layer 350 to the outer portion (vascular scaffold) of the stent. The barrier layer 352 may also control or prevent the diffusion of water or other biological fluids from outside of the stent into the therapeutic layer 350 retained by the hole in the stent. The barrier layer 352 may be biodegradable or erodible material comprising polymer to provide a delayed release of the enclosed therapeutic layer 350. The therapeutic layer 350 may be a drug or alternatively may be a mixture of drug and polymer to further delay or control the elution or release rate of the therapeutic material.

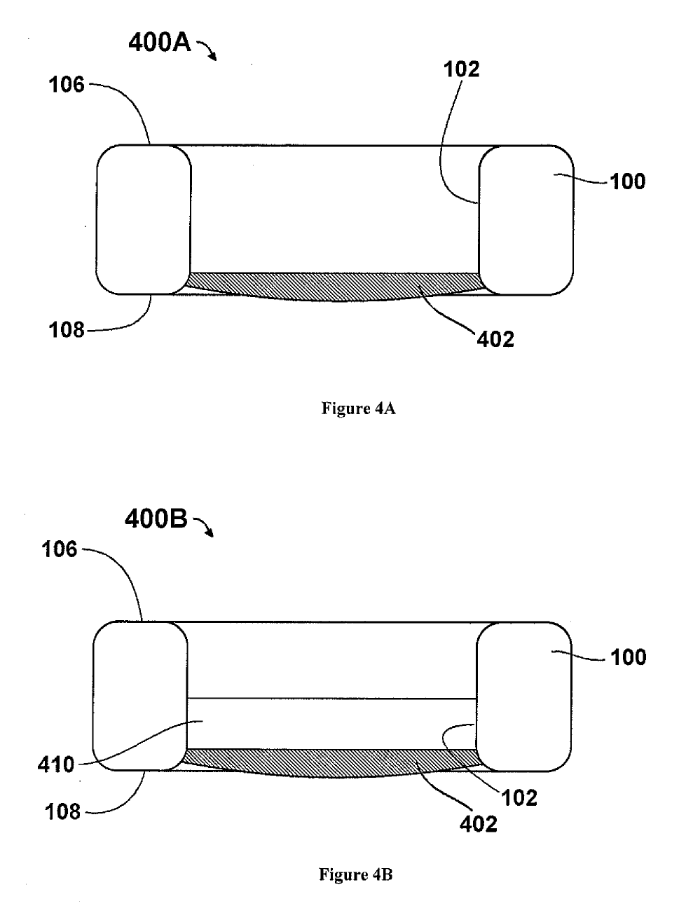

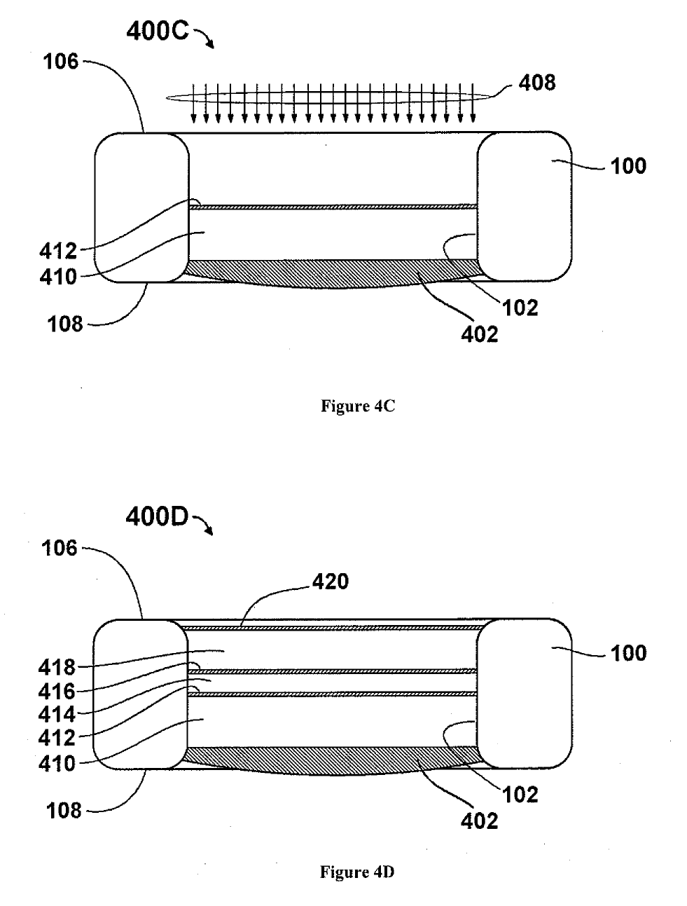

FIG. 4A shows sectional view 400A of a strut of a stent illustrating a step in the formation of a drug-loaded through-hole in a stent 100 according to an embodiment of the invention. A stent 100 has a through-hole 102. The stent has an inner surface 108 forming the lumen of the stent and has an outer surface 106 forming the vascular scaffold portion of the stent. As a step in the embodiment of the invention, a barrier layer 402 is deposited on the inner surface 108 of the stent 100 according to known technology. The barrier layer 402 may consist of polymer or of other biocompatible barrier material.

FIG. 4B shows sectional view 400B of a strut of a stent illustrating a step in the formation of a drug-loaded through-hole in a stent 100 following the step shown in FIG. 4A. In the step shown in FIG. 4B, a drug 410 is deposited in the hole 102 in the stent 100. The deposition of the drug 410 may be by any of numerous methods, including spraying, dipping, electrostatic deposition, ultrasonic spraying, vapor deposition, or preferably by discrete droplet-on-demand fluid jetting technology. When spraying, dipping, electrostatic deposition, ultrasonic spraying, vapor deposition, or similar techniques are employed, a conventional masking scheme can be beneficially employed to limit deposition to the hole or to several or all of the holes in a stent. Discrete droplet-on-demand fluid-jetting is a preferred deposition method because it provides the ability to introduce precise volumes of liquid drugs or drugs-in-solution into precisely programmable locations. Discrete droplet-on-demand fluid jetting may be accomplished using commercially available fluid-jet print head jetting devices as are available (for example, not limitation) from MicroFab Technologies, Inc., of Plano, Tex. When the drug 410 is a liquid or a drug-in-solution, it is preferably dried or otherwise hardened before proceeding to the next step. The drying or hardening step may include baking, low temperature baking, or vacuum evaporation, as examples.

FIG. 4C shows sectional view 400C of a strut of a stent illustrating a step in the formation of a drug-loaded through-hole in a stent 100 following the step shown in FIG. 4B. In the step shown in FIG. 4C, the drug 410 deposited in the hole 102 in the stent 100 is irradiated by an ion beam, preferably GCIB 408 to form a thin barrier layer 412 by modification of a thin upper region of the drug 410. The thin barrier layer 412 consists of drug 410 modified to densify, carbonize or partially carbonize, denature, cross-link, or polymerize molecules of the drug in the thin uppermost layer of the drug 410. The thin barrier layer 412 may have a thickness on the order of about 10 nanometers or even less. In modifying the surface a GCIB 408 comprising preferably argon cluster ions or cluster ions of another inert gas is employed. The GCIB 408 is preferably accelerated with an accelerating potential of from 5 kV to 50 kV or more. The coating layer is preferably exposed to a GCIB dose of at least about 1×10¹³ gas cluster ions per square centimeter. By selecting the dose and/or accelerating potential of the GCIB 408, the characteristics of the thin barrier layer 412 may be adjusted to permit control of the release or elution rate and/or the rate of inward diffusion of water and/or other biological fluids when the stent 100 is implanted and expanded. In general, increasing acceleration potential increases the thickness of the thin barrier layer that is formed, and modifying the GCIB dose changes the nature of the thin barrier layer by changing the degree of cross linking, densification, carbonization, denaturization, and/or polymerization that results. This provides means to control the rate at which drug will subsequently release or elute through the barrier and/or the rate at which water and/or biological fluids my diffuse into the drug from outside.

FIG. 4D shows sectional view 400D of a strut of a stent illustrating a drug-loaded through-hole in a stent 100 following the step shown in FIG. 4C. In FIG. 4D, the steps of depositing a drug and using GCIB irradiation to form a thin barrier layer in the surface of the drug has been repeated (for example) twice more beyond the stage shown in FIG. 4C. FIG. 4D shows the additional layers of drugs (414 and 418) and the additional GCIB-formed thin barrier layers 416 and 420. The drugs 410, 414, and 418 may be the same drug material or may be different drugs with different therapeutic modes. The thicknesses of the layers of drugs 410, 414, and 418 are shown to be different, indicating that different drug doses may be deposited in each individual layer. Alternatively, the thicknesses (and doses) may be the same in some or all layers. The properties of each of the thin barrier layers 412, 416, and 420 may also be individually adjusted by selecting GCIB properties at each barrier layer formation irradiation step by controlling the GCIB properties as discussed above. Although FIG. 4D shows a hole loaded with three layers of drugs, there is complete freedom within the constraints of the hole depth and drug deposition capabilities to utilize from one to a very large number of layers all within the spirit of the invention. The very thin barrier layers that can be formed by GCIB processing and the ability to deposit very small volumes of drug by, for example, discrete droplet-on-demand fluid-jetting technology, make many tens or even hundreds of layers possible. Each drug layer may be different or similar drug materials, may be mixtures of compatible drugs, may be larger or smaller volumes, etcetera, providing great flexibility and control in the therapeutic effect of the drug delivery system and in tailoring the sequencing and elution rates of one or more drugs.

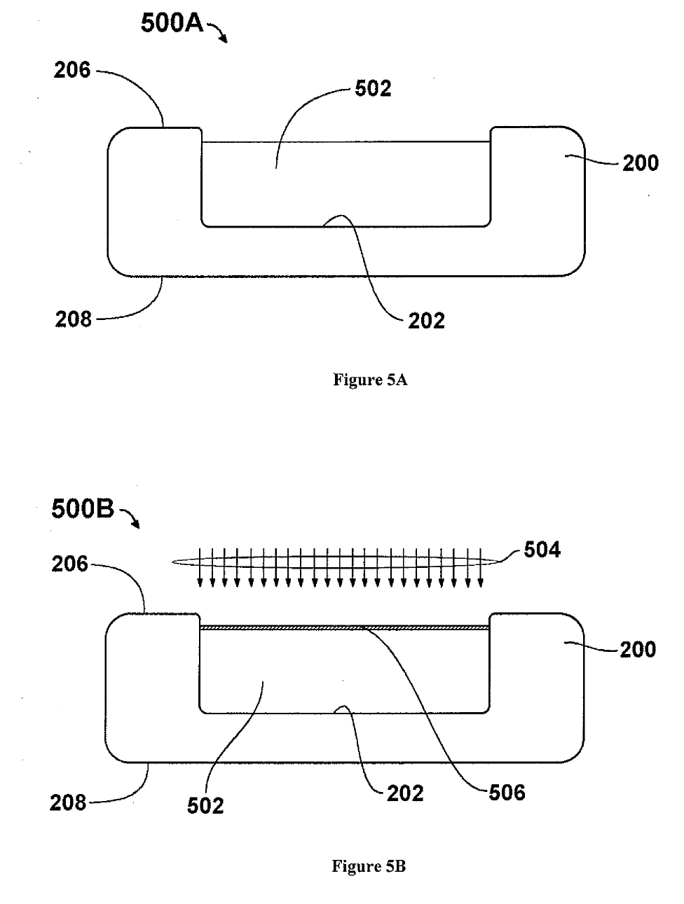

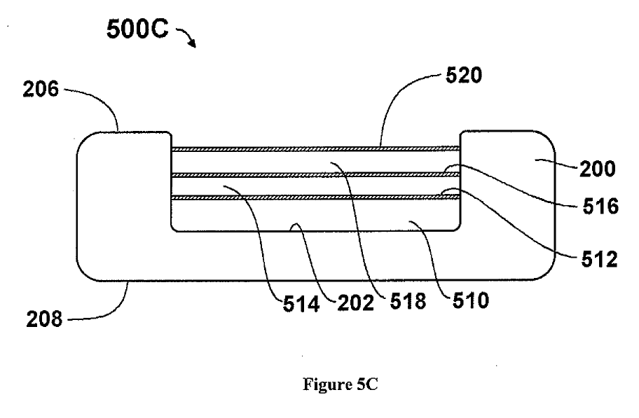

The drug delivery system shown in FIG. 4D is an improvement over prior art systems, but it suffers from the fact that it utilizes a conventional barrier layer 402, that may consist of polymer or of other biocompatible barrier material. In the case of a stent, for example, it is generally not convenient to form a barrier layer by GCIB processing in the interior (lumen) surface of an unexpanded stent. Thus conventional barrier layer 402 is generally required. Use of polymers may be avoided by employing other biocompatible materials for formation of the barrier layer 402; however even so, there is risk of subsequent flaking of the material resulting in its undesired release in situ. FIGS. 5A, 5B, and 5C show another embodiment of the present invention that avoids the undesirable need to use conventional barrier materials.

FIG. 5A shows sectional view 500A of a strut of a stent illustrating a step in the formation of a drug-loaded blind-hole in a stent 200 according to an embodiment of the invention. A stent 200 has a blind-hole 202. The stent has an inner surface 208 forming the lumen of the stent and has an outer surface 206 forming the vascular scaffold portion of the stent. As a step in the embodiment of the invention, a drug 502 is deposited in the hole 202 in the stent 200. Not shown, and optionally, a GCIB cleaning process may be employed to clean the surfaces of the hole 202 prior to depositing drug 502 in the hole 202. The deposition of the drug 502 may be by any of the above-discussed methods. Discrete droplet-on-demand fluid jetting is a preferred deposition method because it provides the ability to introduce precise volumes of liquid drugs or drugs-in-solution into precisely programmable locations. When the drug 502 is a liquid or a drug-in-solution, it is preferably dried or otherwise hardened before proceeding to the next step. The drying or hardening may include baking, low temperature baking, or vacuum evaporation, as examples.

FIG. 5B shows sectional view 500B of a strut of a stent illustrating a step in the formation of a drug-loaded blind-hole in a stent 200 following the step shown in FIG. 5A. In the step shown in FIG. 5B, the drug 502 deposited in the hole 202 in the stent 200 is irradiated by an ion beam, preferably GCIB 504 to form a thin barrier layer 506 by modification of a thin upper region of the drug 502. The thin barrier layer 506 consists of drug 502 modified to densify, carbonize or partially carbonize, denature, cross-link, or polymerize molecules of the drug in the thin uppermost layer of the drug 502. The thin barrier layer 506 may have a thickness on the order of about 10 nanometers or even less. In modifying the surface, a GCIB 504 comprising preferably argon cluster ions or cluster ions of another inert gas is employed. The GCIB 504 is preferably accelerated with an accelerating potential of from 5 kV to 50 kV or more. The coating layer is preferably exposed to a GCIB dose of at least about 1×10¹³ gas cluster ions per square centimeter. By selecting the dose and/or accelerating potential of the GCIB 504, the characteristics of the thin barrier layer 506 may be adjusted to permit control of the elution rate and/or the rate of inward diffusion of water and/or other biological fluids when the stent 200 is implanted and expanded. In general, increasing acceleration potential increases the thickness of the thin barrier layer that is formed, and modifying the GCIB dose changes the nature of the thin barrier layer by changing the degree of cross linking, densification, carbonization, denaturization, and/or polymerization that results. This provides means to control the rate at which drug will subsequently release or elute through the barrier and/or the rate at which water and/or biological fluids my diffuse into the drug from outside.

FIG. 5C shows sectional view 500C of a drug-loaded blind-hole in a stent 200 having multiple drug layers, according to an embodiment of the invention. The steps of depositing a drug and using ion beam irradiation to form a thin barrier layer in the surface of the drug has been as described above for FIGS. 5A and 5B have been applied (for example) three times in succession, forming a blind-hole 202 loaded with three drugs 510, 514, and 518, each having a thin barrier layer 512, 516, and 520 having been formed by ion beam, preferably GCIB, irradiation. The drugs 510, 514, and 518 may be the same drug material or may be different drugs with different therapeutic modes. The thicknesses of the layers of drugs 510, 514, and 518 are shown to be different, indicating that different drug doses may be deposited in each individual layer. Alternatively, the thicknesses (and doses) may be the same in some or all layers. The properties of each of the thin barrier layers 512, 516, and 520 may also be individually adjusted by controlling ion beam properties at each barrier layer formation irradiation step by controlling the GCIB properties as discussed above. Although three layers of drugs are shown, there is complete freedom within the constraints of the hole depth and drug deposition capabilities to utilize from one to a very large number of layers all within the spirit of the invention.

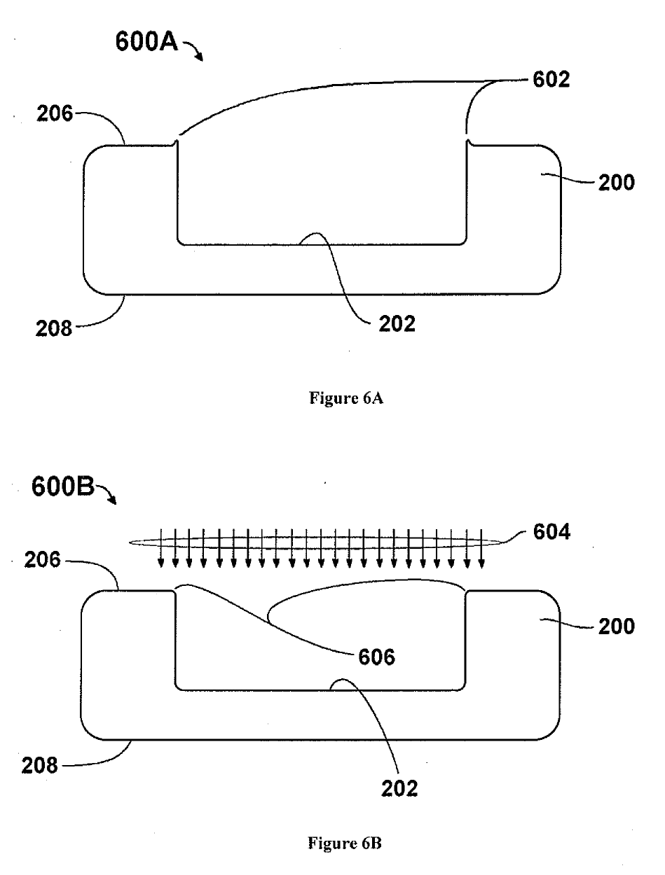

FIG. 6A shows a cross section view 600A of a portion of a blind-hole in an implantable medical device (a stent 200, for example), wherein the hole 202 has been formed by laser machining and has a resulting sharp or (as shown) burred edge 602 resulting from the machining process. In most cases such an edge or burr is undesirable in an implantable medical device. GCIB processing can be advantageously employed to remove such burr or sharp edge prior to loading the hole with a drug and forming a thin barrier layer (as described above).

FIG. 6B shows a cross section view 600B of the hole 202 in stent 200 processed by irradiation with a GCIB 604 to remove the sharp or burred edge 602 by GCIB processing, forming a smooth edge 606. A GCIB 604 comprising preferably argon or nitrogen cluster ions or cluster ions of another inert gas or oxygen cluster ions is employed, The GCIB 604 is preferably accelerated with an accelerating potential of from 5 kV to 50 kV or more. The coating layer is preferably exposed to a GCIB dose of from about 1×10¹⁵ to about 1×10¹⁷ gas cluster ions per square centimeter. By selecting the dose and/or accelerating potential of the GCIB 504, the etching characteristics of the GCIB 604 are adjusted to control the amount of etching and smoothing performed in forming smoothed edge 606. In general, increasing acceleration potential and or increasing the GCIB dose increases the etching rate.

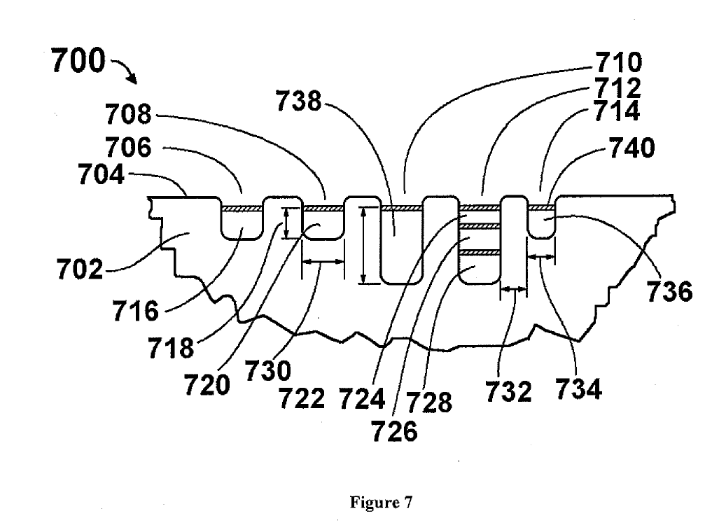

FIG. 7 shows a cross sectional view 700 of the surface 704 of a portion 702 of a non-polymer implantable medical device having a variety of drug-loaded holes 706, 708, 710, 712, and 714 pointing out the diversity and flexibility of the invention. The implantable medical device could, for example, be any of a vascular stent, an artificial joint prosthesis, a cardiac pacemaker, or any other implantable non-polymer medical device and need not necessarily be a thin-walled device like a vascular or coronary stent, The holes all have thin barrier layers 740 formed according to the invention on one or more layers of drug in each hole. For simplicity, not all of the thin barrier layers in FIG. 7 are labeled with reference numerals, but hole 714 is shown containing a first drug 736 covered with a thin barrier layer 740 (only thin barrier layer 740 in hole 714 is labeled with a reference numeral, but each cross-hatched region in FIG. 7 indicates a thin barrier layer, and all will hereinafter be referred to by the exemplary reference numeral 740). Hole 706 contains a second drug 716 covered with a thin barrier layer 740. Hole 708 contains a third drug 720 covered with a thin barrier layer 740. Hole 710 contains a fourth drug 738 covered with a thin barrier layer 740. Hole 712 contains fifth, sixth, and seventh drugs 728, 726, and 724, each respectively covered with a thin barrier layer 740. Each of the respective drugs 716, 720, 724, 726, 728, 736, and 738 may be selected to be a different drug material or may be the same drug materials in various combinations of different or same. Each of the thin barrier layers 740 may have the same or different properties for controlling elution or release rate and/or for controlling the rate of inward diffusion of water or other biological fluids according to ion beam (preferably GCIB) processing principles discussed herein above. Holes 706 and 708 have the same widths and fill depth 718, and thus hold the same volume of drugs, but the drugs 716 and 720 may be different drugs for different therapeutic modes. The thin barrier layers 740 corresponding respectively to holes 706 and 708 may have either same or differing properties for providing same or different elution, release, or inward diffusion rates for the drugs contained in holes 706 and 708. Holes 708 and 710 have the same widths, but differing fill depths, 718 and 722 respectively, thus containing differing drug loads corresponding to differing doses. The thin barrier layers 740 corresponding respectively to holes 708 and 710 may have either same or differing properties for providing same or different elution, release, or inward diffusion rates for the drugs contained in holes 708 and 710. Holes 710 and 712 have the same widths 730, and have the same fill depths 722, thus containing the same total drug loads, but hole 710 is filled with a single layer of drug 738, while hole 712 is filled with multiple layers of drug 724, 726, and 728, which may each be the same or different volumes of drug representing the same or different doses and furthermore may each be different drug materials for different therapeutic modes. Each of the thin barrier layers 740 for holes 710 and 712 may have the same or different properties for providing same or different elution, release, or inward diffusion rates for the drugs contained in the holes. Holes 708 and 714 have the same fill depths 718, but have different widths and thus contain different volumes and doses of drugs 720 and 736. The thin barrier layers 740 corresponding respectively to holes 708 and 714 may have either same or differing properties for providing same or different elution, release, or inward diffusion rates for the drugs contained in holes 708 and 714. The overall hole pattern on the surface 704 of the implantable medical device and the spacing between holes 732 may additionally be selected to control the spatial distribution of drug dose across the surface of the implantable medical device. Thus there are many flexible options in the application of the invention for controlling the types and doses and dose spatial distributions and temporal release sequences and release rates and release rate temporal profiles of drugs delivered by the drug delivery system of the invention.

Although the invention has been described with respect to various embodiments, it should be realized this invention is also capable of a wide variety of further and other embodiments within the spirit and scope of the invention and the appended claims.

It is Claimed:

1. A medical device having a surface adapted for delivering one or more drugs, comprising:
one or more holes in the medical device surface containing the one or more drugs; and
at least one barrier layer comprising a modified drug on at least one drug surface of the one or more drugs contained in the one or more holes.

2. The medical device of claim 1, wherein the at least one or more barrier layers:
control at least one release rate of the one or more drugs;
control at least one elution rate of the one or more drugs; or
control at least one inward diffusion rate of a fluid into at least one drug contained in at least one hole.

3. The medical device of claim 1, wherein the one or more holes are disposed on the medical device surface in a predetermined pattern to spatially distribute the one or more drugs on the medical device surface according to a predetermined distribution plan.

4. The medical device of claim 1, wherein:
a first number of the one or more holes contain a first drug and are arranged in a first pattern;
a second number of the one or more holes contain a second drug and are arranged in a second pattern; and
wherein, the first and second patterns are predetermined to spatially distribute the first and second drugs according to predetermined distribution plans for each drug.

5. The medical device of claim 1, wherein:
a first number of the one or more holes contain a first quantity of a first drug and are arranged in a first pattern;
a second number of the one or more holes contain a second quantity of the drug and are arranged in a second pattern; and
wherein, the first and second patterns are predetermined to spatially distribute the first drug according to predetermined dose distribution plan for the first drug.

6. The medical device of claim 1, wherein at least one of the one or more holes contains a first quantity of a first drug, said first drug overlaid by a first barrier layer comprising modified first drug, said first barrier layer overlaid by a second quantity of a second drug, said second drug overlaid by a second barrier layer comprising modified second drug.

7. The medical device of claim 6, wherein:
the first drug and the second drug are the same drug or different drugs;
the first barrier layer and the second barrier layer are constructed to control a temporal release profile of the first and second drugs.

8. The medical device of claim 1, wherein the medical device is any of:
a vascular stent;
a coronary stent;
an artificial joint prosthesis;
an artificial joint prosthesis component; or
a coronary pacemaker;

9. The medical device of claim 1, wherein the at least one barrier layer comprising modified drug is selected from the group consisting of:
cross-linked drug molecules;
a densified drug;
a carbonized organic drug material;
a polymerized drug;
a denaturized drug; and
combinations thereof.

10. The medical device of claim 1, wherein at least one barrier layer comprises a biologically active material.

11. A method of modifying a surface of a medical device comprising the steps:
forming one or more holes in the surface of the medical device;
first loading at least one of the one or more holes with a first drug; and
first irradiating an exposed surface of the first drug in at least one loaded hole with a first ion beam to form a first barrier layer at the exposed surface.

12. The method of claim 11, wherein the first ion beam is a first gas cluster ion beam.

13. The method of claim 11, further comprising the steps, prior to the loading step:
forming a second ion beam that is a second gas cluster ion beam; and
second irradiating at least a portion of the one or more holes of the medical device with the second ion beam to:
clean the at least a portion of the holes; and/or remove a sharp or burred edge on the at least a portion of the holes.

14. The method of claim 11, wherein the first irradiating step forms the first barrier layer by modifying the first drug at the exposed surface by:
cross-linking first drug molecules;
densifying the first drug;
carbonizing the first drug;
polymerizing the first drug; or
denaturing the first drug.

15. The method of claim 11, wherein the first loading step comprises introducing the first drug into the one or more holes by:
spraying;
dipping;
electrostatic deposition;
ultrasonic spraying;
vapor deposition; or
discrete droplet-on-demand fluid jetting.

16. The method of claim 15, wherein the first loading step further comprises employing a mask to control which of the at least one or more holes are loaded with the first drug.

17. The method of claim 11, wherein the first barrier layer controls a rate of inward diffusion of a fluid into the at least one loaded hole.

18. The method of claim 11, wherein the one or more holes are disposed on the surface in a predetermined pattern to distribute the first drug on the surface according to a predetermined distribution plan.

19. The method of claim 11, further comprising the step of:
second loading at least one of the one or more holes with a second drug different from the first drug.

20. The method of claim 11, wherein at least one of the one or more holes is loaded with a first quantity of the first drug that differs from a second quantity of the first drug loaded in at least another of the one or more holes.

21. The method of claim 11, wherein the first loading step does not completely fill the at least one hole, further comprising the steps of:
second loading the at least one incompletely filled hole with a second drug overlying the first barrier layer; and
third irradiating an exposed surface of the second drug in at least one second loaded hole with a third ion beam to form a second barrier layer at the exposed surface of the second drug in the at least one second loaded hole.

22. The method of claim 21, wherein the first barrier layer and the second barrier layer have different properties for differently controlling elution rates of the first and second drugs.

23. The method of claim 21, wherein the third ion beam is a third gas cluster ion beam.

24. The method of claim 11, wherein the hole forming step comprises forming one or more holes by laser machining or by focused ion beam machining.
